(19) United States
(12) Patent Application Publication
(10) Pub. No.: US 2025/0241847 A1
(43) Pub. Date: Jul. 31, 2025

(54) FORMULATIONS FOR AN ANTI-BRUISING SERUM AND RELATD METHODS OF MANUFACTURE

(71) Applicant: Tiffany Pompa, Galloway, NJ (US)

(72) Inventor: Tiffany Pompa, Galloway, NJ (US)

(21) Appl. No.: 18/831,078

(22) Filed: Jun. 14, 2024

Related U.S. Application Data
(63) Continuation-in-part of application No. 18/445,771, filed on Jan. 29, 2024.

(51) Int. Cl. A61K 8/9789; A61K 8/65; A61K 8/67; A61K 8/73; A61Q 19/00

(52) U.S. Cl. CPC A61K 8/9789; A61K 8/65; A61K 8/676; A61K 8/735; A61Q 19/00; A61K 2800/30

(57) ABSTRACT

A method for manufacturing an anti-bruising formulation comprises admixing therapeutically active agents including at least one skin hydrating agent, at least one moisturizer, at least one anti-inflammatory agent, and at least one skin pigment reducing agent.

### RELATED APPLICATIONS

This application is a continuation-in-part and claims priority to U.S. patent application Ser. No. 18/445,771 filed on Jan. 20, 2024 entitled “A Formulation for an Anti-Bruising Serum”.

### BACKGROUND OF THE INVENTION

Skin bruising can be caused by a plurality of factors including, but not limited to, trauma like surgery or injuries, medications, aging skin, inadequate moisture, loss of skin integrity and decreased fat. “Ecchymosis” is the medical term for a bruise. A bruise, or contusion, is skin discoloration from damaged, leaking blood vessels underneath your skin. Even though there's blood pooling underneath your skin, you won't have any external bleeding unless your skin breaks open. The collection of blood makes a bruise visible. Bruises form a mark on your skin that ranges in color from black, blue, purple, brown or yellow.

Bruises are very common and will affect everyone at some point in their life. Bruises can occur from a fall, accident, sports injury or medical procedure. People older than 65 years are more likely to bruise, along with women and people assigned female at birth.

There are some bleeding disorders and medical conditions that may make you more prone to bruising, including people who: suffer from cancer or liver disease; bruise easily; take medications to thin blood or stop clotting, such as aspirin or blood thinners; regularly take nonsteroidal anti-inflammatory drugs (NSAIDs) for pain relief, including ibuprofen or naproxen; have a bleeding disorder such as hemophilia, von Willebrand disease or another blood clotting condition; Experience a low blood platelet count (thrombocytopenia); and have a vitamin C or vitamin K deficiency.

Your body's blood cells repair damaged blood vessels to help you heal. However, healing time takes a minimum of at least two or more weeks and the bruises can cause numerous emotional issues as well as physical pain. The visible appearance of bruises can destroy an individual's confidence and can bring about unwanted questions, intruding into one's privacy and causing embarrassment. In view of the above, the present invention provides for a formulation and a related method of manufacture for a formulation that is an anti-bruising serum.

### SUMMARY OF THE INVENTION

The formulation of the present invention can repair skin bruising, moisturize the skin, improve skin integrity, reduce skin coloration, induce tissue regeneration, provide anti-inflammatory benefits, reduce signs of aging, provide pain relief, reduce discomfort of neuropathy, improve skin tone, stimulate healing, and prevent the formation of new bruises. In an embodiment, the formulation is in the form of a serum which must first be shaken, prior to use, as there is separation between the oil and the other ingredients. For application, a small amount of the serum is placed and smoothed over the bruised area of the skin. No rubbing is required. The natural antioxidants help in the balance of the wound healing to speed up the recovery process and stimulate collagen production.

In one embodiment, the present invention relates to a formulation comprising therapeutically active agents, and the therapeutically active agents are selected from the group consisting of at least one skin hydrating agent, at least one moisturizer, at least one anti-inflammatory agent, and at least one skin pigment reducing agent.

In another embodiment, the skin hydrating agent is selected from the group consisting of hyaluronic acid, aloe vera, cassia angustifolia seed polysaccharide, centella asiatica, green tea extract, honey, glycerin, glycerol, N-acetylglucosamine, polyglutamic acid, Beta glucan, retinol, vitamin E, ceramides, and combination and mixtures thereof.

In yet another embodiment, the moisturizer is selected from the group consisting of vegan collagen, glycolic acid, copper peptides, amino acids, rose hip, carrot seed, chamomile, eucalyptus, aloe vera, ginseng, and combination and mixtures thereof.

In still another embodiment, the anti-inflammatory agent is selected from the group consisting of arnica herbal extract, bromelain, vitamin K, quercetin, frankincense, rosemary, lavender, helichrysum, geranium, lemongrass, and combination and mixtures thereof.

In still yet another embodiment, the skin pigment reducing agent is selected from the group consisting of vitamin C, niacinamide, lactic acid, mandelic acid, tranexamic acid, alpha arbutin, azelaic acid, retinol, and combination and mixtures thereof.

In a further embodiment, the anti-inflammatory agent is infused in oil. In yet a further embodiment, the oil is selected from the group consisting of olive oil, grapeseed oil, cocoa butter, shea butter, coconut oil, wheat germ oil, and combination and mixtures thereof. In still a further embodiment, the formulation is in the form of a serum.

In still yet a further embodiment, the formulation further comprises an additive, said additive is selected from the group consisting of at least one preservative, at least one thickening agent, at least one colorant and at least one fragrance, and combinations and mixtures thereof. In another further embodiment, the preservative is selected from the group consisting of an organic preservative, inorganic preservative, radish root ferment filtrate, and combination and mixtures thereof.

In another embodiment, the skin hydrating agent is from about 50% to about 70.0% by weight of said formulation. In yet another embodiment, the moisturizer is from about 1.0% to about 8% by weight of said formulation. In still another embodiment, the anti-inflammatory agent is from about 25% to about 40% by weight of said formulation. In still yet another embodiment, the skin pigment reducing agent is from about 0.5% to about 5% by weight of said formulation.

In a further embodiment, the present invention relates to a formulation comprising therapeutically active agents, and the therapeutically active agents are selected from the group consisting of hyaluronic acid, vegan collagen, arnica herbal extract, and vitamin C. In another further embodiment, the arnica herbal extract is infused in oil. In yet another further embodiment, the hyaluronic acid functions as a skin hydrating agent, the vegan collagen functions as a moisturizer, the arnica herbal extract functions as an inflammatory agent, and the vitamin C functions as a skin pigment reducing agent. In still another further embodiment, the formulation further comprises at least one preservative and at least one thickening agent. The preservative is from about 0.5% to about 2.5% by weight of the formulation.

In still yet another further embodiment, the hyaluronic acid is from about 50% to about 70.0% by weight of the formulation, the vegan collagen is from about 1.0% to about 8% by weight of the formulation, the arnica herbal extract is from about 25% to about 40% by weight of the formulation, and the vitamin C is from about 0.5% to about 5% by weight of the formulation.

In another further embodiment, the present invention provides for a formulation for an anti-bruising serum comprising therapeutically active agents, said therapeutically active agents are a combination of Mixture A and B, said Mixture A is selected from the group consisting of hyaluronic acid, vegan collagen, and vitamin C, and said Mixture B is arnica herbal extract. In another embodiment, Mixture A is from about 60% to about 75% by weight of the formulation, and said Mixture B is from about 25% to about 40% by weight of the formulation.

In one embodiment, the present invention relates to a method of manufacturing a serum for preventing and treating the visible appearance of skin bruises, said method comprising: admixing at least one skin hydrating agent, at least one moisturizer, and at least one skin pigment reducing agent to form Mixture A; infusing at least one anti-inflammatory agent in an oil to form Mixture B; and admixing Mixture A and Mixture B to form a serum.

In another embodiment, hyaluronic acid functions as said at least one skin hydrating agent, vegan collagen functions as said at least one moisturizer, arnica herbal extract functions as said at least one inflammatory agent, vitamin C functions as said at least one skin pigment reducing agent, and said olive oil is said oil.

In yet another embodiment, the method further comprises admixing at least one preservative. In still another embodiment, mixture A is from 60% to about 75% by weight of said formulation. In still yet another embodiment, Mixture B is from 25% to about 40% by weight of said formulation.

In a further embodiment, the method further comprises the step of: admixing an additive, said additive is selected from the group consisting of at least one preservative, at least one thickening agent, at least one colorant and at least one fragrance, and combinations and mixtures thereof. In yet a further embodiment, the preservative is selected from the group consisting of an organic preservative, inorganic preservative, radish root ferment filtrate, and combination and mixtures thereof.

In still a further embodiment, the method of manufacturing therapeutically active agents for a formulation that prevents and treats the visible appearance of skin bruises, said method comprising: admixing at least one skin hydrating agent, at least one moisturizer, at least one anti-inflammatory agent, and at least one skin pigment reducing agent.

In still yet a further embodiment, the skin hydrating agent is selected from the group consisting of hyaluronic acid, aloe vera, cassia angustifolia seed polysaccharide, centella asiatica, green tea extract, honey, glycerin, glycerol, N-acetylglucosamine, polyglutamic acid, Beta glucan, retinol, vitamin E, ceramides, and combination and mixtures thereof.

In another further embodiment, the moisturizer is selected from the group consisting of collagen, glycolic acid, copper peptides, amino acids, rose hip, carrot seed, chamomile, eucalyptus, aloe vera, ginseng, and combination and mixtures thereof.

In yet another further embodiment, the anti-inflammatory agent is selected from the group consisting of arnica herbal extract, Arnica chamissonis, Arnica montana, bromelain, vitamin K, quercetin, frankincense, rosemary, lavender, helichrysum, geranium, lemongrass, and combination and mixtures thereof.

In still another further embodiment, the skin pigment reducing agent is selected from the group consisting of vitamin C, niacinamide, lactic acid, mandelic acid, tranexamic acid, alpha arbutin, azelaic acid, retinol, and combination and mixtures thereof.

In still yet a further embodiment, the method further comprises the step of: infusing said at least one anti-inflammatory agent in oil prior to admixing with said other therapeutically active agents for said formulation. In another embodiment, the oil is selected from the group consisting of olive oil, grapeseed oil, cocoa butter, shea butter, coconut oil, wheat germ oil, and combination and mixtures thereof.

In yet another embodiment, the method further comprises the step of: incorporating said therapeutically active agents with at least one humectant to form a serum. In still another embodiment, the humectant is selected from the group consisting of urea, glycerin, hyaluronic acid, aloe vera, honey, butylene glycol, panthenol, seaweed, cocoa butter, arnica extract, collagen, paraffin, propylene glycol, sorbitol, lactic acid, alpha hydroxycarboxylic acid, sodium, betaine, sodium hyaluronate, dimethicone, mineral oil, polyethylene glycol, and mixtures and combinations thereof.

In still another embodiment, the method further comprises the step of: incorporating said therapeutically active agents with at least one emulsifier to form a cream. In still yet another embodiment, the emulsifier is selected from the group consisting of xanthan gum, stearic acid, glyceryl stearate, beeswax, stearic acid, lecithin, cetearyl alcohol, cetearyl olivate, sorbitan olivate, and combinations and mixtures thereof.

In a further embodiment, the method further comprises the step of: admixing an additive, said additive is selected from the group consisting of at least one preservative, at least one thickening agent, at least one colorant and at least one fragrance, and combinations and mixtures thereof.

In another further embodiment, the preservative is selected from the group consisting of an organic preservative, inorganic preservative, radish root ferment filtrate, and combination and mixtures thereof, said at least one preservative is from about 0.5% to about 2.5% by weight of the formulation.

In a further embodiment, the skin hydrating agent is from about 50% to about 70.0% by weight of the formulation, the moisturizer is from about 1.0% to about 8% by weight of the formulation, the anti-inflammatory agent is from about 25% to about 40% by weight of the formulation, and the skin pigment reducing agent is from about 0.5% to about 5% by weight of the formulation.

### DETAILED DESCRIPTION OF THE INVENTION

As required, detailed embodiments of the present invention are disclosed herein; however, it is to be understood that the disclosed embodiments are merely exemplary of the invention that may be embodied in various forms. The figures are not necessarily to scale, some features may be exaggerated to show details of particular components. Therefore, specific structural and functional details disclosed herein are not to be interpreted as limiting, but merely as a basis for the claims and as a representative basis for teaching one skilled in the art to variously employ the present invention.

The present invention provides a formulation that functions to improve the visible appearance of bruises, to regenerate dermal tissues, to reduce discoloration of the skin caused by bruises, to prevent new bruise formation, to improve skin moisture and integrity, to reduce signs of aging, to stimulate healing, to address pain relief and inflammation, and to improve skin tone and texture. The presently claimed invention provides for a formulation that comprises therapeutically active agents including at least one skin hydrating agent, at least one moisturizer, at least one anti-inflammatory agent, and at least one skin pigment reducing agent. For purposes of this invention, the formulation and its therapeutically active agents are not limited to the above and below mentioned functions.

### Hyaluronic Acid as a Skin Hydrating Agent:

In one embodiment, the skin hydrating agent is hyaluronic acid which is also known as hyaluronan or hyaluronate. It is a gooey, slippery substance that your body produces naturally. Scientists have found hyaluronic acid throughout the body, especially in eyes, joints and skin. It makes your skin flexible. Hyaluronic acid helps skin stretch and flex and reduces wrinkles and lines in the skin. Hyaluronic acid is also proven to help wounds heal faster and can reduce scarring. Hyaluronic acid is often produced by fermenting certain types of bacteria. Rooster combs (the red, Mohawk-like growth on top of a rooster's head and face) are also a common source.

Hyaluronic acid belongs to a type of long chain-like molecules called polymers. The chain has plenty of spots on it where other chemical compounds (like water, for example) can latch on. That's why a quarter-teaspoon of hyaluronic acid can hold about one and a half gallons of water, making it the best polymer, natural or artificial, for absorbing water (and a key ingredient in moisturizing products).

Because it has lots of space for other molecules to latch on, hyaluronic acid is great for transporting other molecules throughout your body. It also has the ability to attach itself to cells, which is why targeted delivery of medications using hyaluronic acid is a major topic of study.

Hyaluronic acid's chain-like structure also means it can act like a scaffold structure, allowing tissues to grow. This is a key step in how wounds heal on your body. Scientists have also found hyaluronic acid in human embryos and are studying what role hyaluronic acid plays in reproduction and development.

Long-term use of hyaluronic acid serum on your skin or in a supplement taken by mouth can improve overall skin health. It's also great for helping improve overall skin flexibility and elasticity (meaning it makes your skin more stretchy and soft).

Research shows that hyaluronic acid is safe to use. Reactions or adverse effects from hyaluronic acid are rare, and it's safe to use if you're pregnant or nursing.

For purposes of this invention, there are various substitutes for hyaluronic acid including, but not limited to, aloe vera, cassia angustifolia seed polysaccharide, centella asiatica, green tea extract, honey, glycerin, glycerol, N-acetylglucosamine, polyglutamic acid, Beta glucan, retinol, vitamin E, ceramides, and other skin hydrating agents.

### Collagen as a Skin Moisturizer:

Collagen is known for having great moisturizing benefits when applied topically. However, the collagen molecules are large and generally do not penetrate the upper layer of the skin. It has to be broken down into smaller peptides or amino acids, which then in turn can be absorbed. For the purpose of this invention, animal or vegan collagen may be used. Collagen causes an increase in fibroblasts and extracellular matrix proteins and a decrease in metalloproteinase. These rising fibroblasts found in the various layers of the human dermis produce a plethora of extracellular matrix proteins that enhance skin health and thus slow skin aging.

Vegan collagen in serums infuse hydration for plump, firm skin, and they also minimize the appearance of pores. This compound is similar to animal-derived collagen in function as well as benefits. Ingestible plant-based collagen is not real collagen, but a collection of vegan ingredients designed to help boost your body's own collagen production.

For purposes of this invention, there are various substitutes for collagen including, but not limited to, glycolic acid, copper peptides, amino acids, rose hip, carrot seed, chamomile, eucalyptus, aloe vera, ginseng, and other skin moisturizers.

### Arnica Herbal Extract as a Anti-Inflammatory Agent:

The anti-inflammatory agent functions to improve blood circulation, to improve healing, to provide anti-inflammation, pain relief and reduction of discomfort caused by neuropathy. In one embodiment, the anti-inflammatory agent is Arnica chamissonis or Arnica montana extract and the extract is infused into oil.

Arnica chamissonis originates from the American West. Once implanted, this beautiful hardy plant will form beautiful covering mats. It is sometimes called “fall grass” . . . arnica is a particularly effective remedy, for external use, to relieve hematomas, inflammations and other sprains.

Arnica montana, also known as wolf's bane, is an herbal medication used topically for pain and inflammation but is of unproven efficacy. It is an extract of the flowering plant Arnica montana, a member of the daisy family

The similarity of the two plant species implies that Arnica chamissonis can be a pharmaceutical substitute for the Arnica montana, i.e., an endangered and endemic plant species in Europe. Chamisso arnica extracts exhibit anti-inflammatory and antiradical activity and possesses high antioxidant properties that might be helpful in preventing or delaying the progress of free radical dependent diseases. The attributes of Arnica chamissonis are mainly related to the content and chemical composition of essential oil.

For purposes of this invention, there are various substitutes for arnica herbal extracts including, but not limited to, bromelain, vitamin K, quercetin, frankincense, rosemary, lavender, helichrysum, geranium, lemongrass, and other anti-inflammatory agents.

### Vitamin C as a Skin Pigment Reducing Agent:

Vitamin C, or L-ascorbic acid, is a water-soluble antioxidant that is a popular cosmeceutical ingredient. Topical application of vitamin C prior to UV exposure confers photoprotection against sunburn and UV-induced immunosuppression. In addition, vitamin C acts as a cofactor for lysyl and prolyl hydroxylase, the enzymes responsible for the synthesis of collagen. Clinical studies confirm that consistent use of topical vitamin C improves skin wrinkling and gives it a more youthful appearance. Vitamin C inhibits tyrosinase and reduces melanin, making it an effective skin-lightening agent. Since it impedes melanin production, vitamin C can actually fade hyperpigmentation. Hyperpigmentation, including sunspots, age spots, and melasma, occurs when melanin is overproduced in certain areas of the skin. It can also happen in areas where acne has healed. Vitamin C inhibits melanin synthesis by downregulating the activity of an enzyme known as tyrosinase. It's widely used in dermatology for reducing pigmentation of hyperpigmented spots on the skin.

Vitamin C is also an effective anti-inflammatory compound and has been evaluated in the postprocedure setting. Clinical studies have demonstrated that topical vitamin C significantly reduces post carbon dioxide (CO2) laser resurfacing erythema. Products containing vitamin C in combination with other antioxidants have been shown to provide added benefits when compared with vitamin C alone.

The skin pigment reducing agent functions to brighten skin tone and texture, to smoothen skin, to minimize signs of aging, and to reduce pigmentation. For purposes of this invention, there are various substitutes for vitamin C including, but not limited to, niacinamide, lactic acid, mandelic acid, tranexamic acid, alpha arbutin, azelaic acid, retinol, and other skin pigment reducing agents.

In one of the embodiments of the present invention, Table 1, set forth below, represents an anti-bruising formulation:

TABLE 1

| Active Agents: | % By Weight of Formulation |
|---|---|
| skin hydrating agent | from about 50% to about 70.0% |
| moisturizer | from about 1.0% to about 8% |
| anti-inflammatory agent | from about 25% to about 40% |
| skin pigment reducing agent | from about 0.5% to about 5% |

In another embodiment, Table 2, set forth below, relates to another anti-bruising formulation:

TABLE 2

| Active Agents: | % By Weight of Formulation |
|---|---|
| hyaluronic acid | from about 50% to about 70.0% |
| vegan collagen | from about 1.0% to about 8% |
| arnica herbal extract | from about 25% to about 40% |
| vitamin C | from about 0.5% to about 5% |
| preservative | from about 0.5% to about 2.5% |

In still another embodiment, Table 3, set forth below, relates to another anti-bruising formulation:

TABLE 3

| Mixture: | % By Weight of Formulation |
|---|---|
| Mixture A | from about 60% to about 75% |
| (hyaluronic acid, vegan collagen, | |
| and vitamin C) | |
| Mixture B | from about 25% to about 40% |
| (arnica herbal extract) | |

Mixture A comprises hyaluronic acid, vegan collagen, and vitamin C and Mixture B comprises arnica herbal extract. As shown in Table 3, the ingredients of Mixture A are first admixed then Mixture B is admixed with Mixture A.

In still yet another embodiment, Table 4, set forth below, relates to another anti-bruising formulation:

TABLE 4

| Mixture: | % By Weight of Formulation |
|---|---|
| Mixture A | from about 60% to about 75% |
| (hyaluronic acid, vegan | |
| collagen, vitamin C and | |
| Leucidal preservative) | |
| Mixture B | from about 25% to about 40% |
| (arnica herbal extract infused in oil) | |

Mixture A comprises hyaluronic acid, vegan collagen, vitamin C and Leucidal preservative; and Mixture B comprises arnica herbal extract infused in oil. As shown in Table 3, the ingredients of Mixture A are first admixed then Mixture B is admixed with Mixture A. In a further embodiment, the arnica herbal extract is infused in oil before being admixed with Mixture A. The ingredient composition of the Leucidal preservative is 37.5% Water; 37.5% Leuconostoc/Radish Root Ferment Filtrate; 20% Lactobacillus; and 5% Cocos Nucifera (Coconut) Fruit Extract.

Among those benefits and improvements that have been disclosed, other objects and advantages of this invention will become apparent from the following description taken in conjunction with the accompanying drawings. The tables and examples constitute a part of this specification and include exemplary embodiments of the present invention and illustrate various objects and features thereof.

Numerous modifications and variations of the present invention are possible in light of the above teachings. It is therefore to be understood that within the scope of the attendant claims attached hereto, this invention may be practiced otherwise than as specifically disclosed herein.

What is claimed is:

1. A method of manufacturing a serum for preventing and treating the visible appearance of skin bruises, said method comprising:
admixing at least one skin hydrating agent, at least one moisturizer, and at least one skin pigment reducing agent to form Mixture A;
infusing at least one anti-inflammatory agent in an oil to form Mixture B; and
admixing Mixture A and Mixture B to form a serum.

2. The method of claim 1 wherein hyaluronic acid functions as said at least one skin hydrating agent, vegan collagen functions as said at least one moisturizer, arnica herbal extract functions as said at least one inflammatory agent, vitamin C functions as said at least one skin pigment reducing agent, and said olive oil is said oil.

3. The method of claim 1 further comprises admixing at least one preservative.

4. The method of claim 1 wherein mixture A is from 60% to about 75% by weight of said formulation.

5. The method of claim 1 wherein Mixture B is from 25% to about 40% by weight of said formulation.

6. The method of claim 1 further comprises the step of: admixing an additive, said additive is selected from the group consisting of at least one preservative, at least one thickening agent, at least one colorant and at least one fragrance, and combinations and mixtures thereof.

7. The method of claim 6 wherein said at least one preservative is selected from the group consisting of an organic preservative, inorganic preservative, radish root ferment filtrate, and combination and mixtures thereof.

8. A method of manufacturing therapeutically active agents for a formulation that prevents and treats the visible appearance of skin bruises, said method comprising:
admixing at least one skin hydrating agent, at least one moisturizer, at least one anti-inflammatory agent, and at least one skin pigment reducing agent.

9. The method of claim 8 wherein said at least one skin hydrating agent is selected from the group consisting of hyaluronic acid, aloe vera, cassia angustifolia seed polysaccharide, centella asiatica, green tea extract, honey, glycerin, glycerol, N-acetylglucosamine, polyglutamic acid, Beta glucan, retinol, vitamin E, ceramides, and combination and mixtures thereof.

10. The method of claim 8 wherein said at least one moisturizer is selected from the group consisting of collagen, glycolic acid, copper peptides, amino acids, rose hip, carrot seed, chamomile, eucalyptus, aloe vera, ginseng, and combination and mixtures thereof.

11. The method of claim 8 wherein said at least one anti-inflammatory agent is selected from the group consisting of arnica herbal extract, Arnica chamissonis, Arnica montana, bromelain, vitamin K, quercetin, frankincense, rosemary, lavender, helichrysum, geranium, lemongrass, and combination and mixtures thereof.

12. The method of claim 8 wherein said skin pigment reducing agent is selected from the group consisting of vitamin C, niacinamide, lactic acid, mandelic acid, tranexamic acid, alpha arbutin, azelaic acid, retinol, and combination and mixtures thereof.

13. The method of claim 8 further comprises the step of: infusing said at least one anti-inflammatory agent in oil prior to admixing with said other therapeutically active agents for said formulation.

14. The method of claim 13 wherein said oil is selected from the group consisting of olive oil, grapeseed oil, cocoa butter, shea butter, coconut oil, wheat germ oil, and combination and mixtures thereof.

15. The method of claim 13 further comprises the step of: incorporating said therapeutically active agents with at least one humectant to form a serum.

16. The method of claim 15 wherein said humectant is selected from the group consisting of urea, glycerin, hyaluronic acid, aloe vera, honey, butylene glycol, panthenol, seaweed, cocoa butter, arnica extract, collagen, paraffin, propylene glycol, sorbitol, lactic acid, alpha hydroxycarboxylic acid, sodium, betaine, sodium hyaluronate, dimethicone, mineral oil, polyethylene glycol, and mixtures and combinations thereof.

17. The method of claim 13 further comprises the step of: incorporating said therapeutically active agents with at least one emulsifier to form a cream.

18. The method of claim 17 wherein said emulsifier is selected from the group consisting of xanthan gum, stearic acid, glyceryl stearate, beeswax, stearic acid, lecithin, cetearyl alcohol, cetearyl olivate, sorbitan olivate, and combinations and mixtures thereof.

19. The method of claim 13 further comprises the step of: admixing an additive, said additive is selected from the group consisting of at least one preservative, at least one thickening agent, at least one colorant and at least one fragrance, and combinations and mixtures thereof.

20. The method of claim 19 wherein said at least one preservative is selected from the group consisting of an organic preservative, inorganic preservative, radish root ferment filtrate, and combination and mixtures thereof, said at least one preservative is from about 0.5% to about 2.5% by weight of said formulation.

21. The method of claim 8 wherein said at least one skin hydrating agent is from about 50% to about 70.0% by weight of said formulation, said at least one moisturizer is from about 1.0% to about 8% by weight of said formulation, said at least one anti-inflammatory agent is from about 25% to about 40% by weight of said formulation, and said at least one skin pigment reducing agent is from about 0.5% to about 5% by weight of said formulation.
